(19) United States
(12) Patent Application Publication
(10) Pub. No.: US 2025/0205299 A1
(43) Pub. Date: Jun. 26, 2025

(54) WATER SOLUBLE TOMATO EXTRACT PROTECTS AGAINST ADVERSE EFFECTS OF AIR POLLUTION

(71) Applicant: Provexis Natural Products Limited, Reading (GB)

(72) Inventors: Bernd MUSSLER, Kaiseraugst (CH); Daniel RAEDERSTORFF, Kaiseraugst (CH); Nathalie RICHARD, Kaiseraugst (CH)

(73) Assignee: Provexis Natural Products Limited, Reading (GB)

(21) Appl. No.: 18/966,432

(22) Filed: Dec. 3, 2024

Related U.S. Application Data
(63) Continuation of application No. 17/140,570, filed on Jan. 4, 2021, now abandoned.
Application No. 17/140,570 is a continuation of application No. 16/346,013, filed on Apr. 29, 2019, now Pat. No. 10,905,733.

(30) Foreign Application Priority Data
Nov. 2, 2016 (EP) 16196794.8

(51) Int. Cl. A61K 36/81; A23L 33/105

(52) U.S. Cl. CPC A61K 36/81; A23L 33/105

(57) ABSTRACT

The present invention relates to compositions comprising a water soluble tomato extract (WSTE) which may be used in maintaining cardiovascular health, lessening the risk of developing cardiovascular health problems, or reducing the likelihood of worsening an existing cardiovascular health problem in a subject exposed, or is at risk of exposure, to particulate air pollution.

### CROSS-REFERENCE TO RELATED APPLICATIONS

This application is a Continuation of U.S. patent application Ser. No. 17/140,570 filed Jan. 4, 2021, which is a Continuation of U.S. patent application Ser. No. 16/346,013 filed Apr. 29, 2019, now issued U.S. Pat. No. 10,905,733, which is a National Phase of International Application No. PCT/EP2017/077993 filed Nov. 2, 2017, which designated the U.S. and that International Application was published under PCT Article 21 (2) in English, the entirety of which is hereby incorporated by reference. This application also includes a claim of priority under 35 U.S.C. § 119 (a) and § 365 (b) to European patent application No. EP 16196794.8 filed Nov. 2, 2016, the entirety of which is hereby incorporated by reference.

### BRIEF DESCRIPTION OF THE INVENTION

This invention relates to the use of a water-soluble tomato extract (“WSTE”) to protect against adverse effects of air pollution on the body's cardiovascular system.

### BACKGROUND OF THE INVENTION

Air pollution comes in many forms. A common type of pollution is referred to as “particulate air pollution”, which contains pollution in the form of soot, gases and other matter which are in the form of tiny particles, termed “respirable particulate matter”. Respirable particulate matter is categorized by size, such as below 10 or 2.5 microns aerodynamic diameter (PM₁₀ or PM_{2.5}, respectively), or as nanoparticles (less than 100 nm diameter, or PM_{0.1}). These particles often come from vehicle emissions, particularly diesel fuel, or from diesel-powered machinery.

It has been shown that particulate matter is able to enter the blood stream and induce cytotoxic and inflammatory responses, and there is a recognized link between exposure to diesel emissions and cardiovascular disease. However, the actual mechanism of how this is accomplished is still not fully understood. See Solomon et al 2013 J. Thromb Haemost 11:325-34; Tabor et al 2016 Particle and Fibre Toxicology 13:6 DOI 10.1186/s12989-016-0116-x; Lucking et al 2008 European Heart J 29:3043-3051; and Hunter et al 2014 Particle and Fibre Technology 11:62 DOI 10.1186/s12989-014-0062-4.

Air pollution is a mixture of particulate matter (PM) and gaseous components. Numerous studies show that exposure to PM air pollution has adverse effect on cardiovascular health (Miller et al. 2012, Pope et al. 2015). Platelet activity/reactivity is linked to an increased risk of cardiovascular diseases especially thrombosis and will also contribute to the development of atherosclerosis. Platelet activity/reactivity can be increased by a number of factors notably air pollution. Thus, PM has been shown to promote arterial thrombosis and atherosclerosis through increased platelet activation.

Not all platelet anti-aggregation agents work via the same pathway, nor are anti-aggregation agents responsive to all aggregation stimuli. For example, clopidrogel (an anti-platelet pharmaceutical used in the secondary prevention of cardiovascular complications of atherosclerosis) can inhibit adenosine 5′-diphosphate (ADP)-induced platelet aggregation but not platelet aggregation induced by collagen or thrombin. (see Weber et al 1999 British J Pharmacologyl26: 415-420). PM may stimulate platelet aggregation via a physical mechanism in addition to a physiological mechanism (such as the mechanism seen in oxygen radical-induced aggregation).

Water soluble tomato extracts which are lycopene-free have been described; see, e.g. WO2010/049707; WO10/049709, and WO99/55350 (all by Provexis Natural Products, Ltd). They are commercially available from DSM Nutritional Products, Switzerland under the registered trademark FRUITFLOW and FRUITFLOW 2. They have been described as having anti-platelet aggregation abilities, which are presumed to be due to the presence of nucleosides and other active agents in the extracts, such as adenosine, caffeic acid derivatives including chlorogenic acid, and flavonoids such as rutin and quercetin-3,4-glycoside.

It would be desirable to have a safe, effective nutraceutical, food or food supplement, nutraceutical or medicament which could help ameliorate the effects of particulate air pollution.

### DETAILED DESCRIPTION OF THE INVENTION

It has been found, in accordance with this invention, that a water soluble tomato extract can protect the cardiovascular system against the adverse effects brought on by exposure to particulate air pollution. Thus, the invention concerns the use of a water soluble tomato extract (WSTE) for the maintenance of a healthy cardiovascular system, and/or to prevent platelet aggregation brought on by interaction with air pollution particulate matter.

According to a first aspect of the invention there is provided a composition comprising a Water soluble tomato extract for use in maintaining cardiovascular health, lessening the risk of developing a cardiovascular health problem, or reducing the likelihood of worsening an existing cardiovascular health problem in a subject who is exposed, or is at risk of exposure, to particulate air pollution. According to another aspect of this invention there is provided a method of maintaining cardiovascular health, lessening the risk of developing a cardiovascular health problem, or reducing the likelihood of worsening an existing cardiovascular health problem in a subject exposed, or is at risk of exposure, to particulate air pollution comprising administering a protective amount of a water soluble tomato extract prior to or concomitant with exposure to air pollution particulate matter.

Another aspect of this invention provides the use of WSTE in the manufacture of a medicament, nutraceutical, food supplement or food for use in maintaining cardiovascular health, lessening the risk of developing a cardiovascular health problem, or reducing the likelihood of worsening an existing cardiovascular health problem in a subject who is exposed, or is at risk of exposure, to particulate air pollution.

Another aspect of this invention provides a dosage form comprising a medicament, nutraceutical, food supplement or food comprising an effective amount of WSTE for protecting against the adverse effects of particulate air pollution.

The inventors believe that WSTE is effective for maintaining cardiovascular health because, as discussed below, WSTE decreases or minimizes the risk of platelet aggregation in a subject exposed to particulate air pollution. Thus, according to another aspect of the invention, there is provided a method of decreasing, or minimizing the risk of, platelet aggregation in a subject exposed to particulate air pollution comprising administering to the subject an effective amount of WSTE prior to or during exposure to particulate air pollution.

### BRIEF DESCRIPTION OF THE FIGURES

FIG. 1 shows the effect of FRUITFLOW on the amount of aggregation (maximal % aggregation) induced by particulate matter. Details are in the Examples.

FIG. 2 shows the effect of FRUITFLOW on the amount of aggregation (maximal % aggregation induced by particulate matter and ADP (adenosine diphosphate)

### DEFINITIONS

As used in the specification and claims, the following definitions apply:

Water Soluble Tomato Extract (“WSTE”); The WISE used in this invention has the following properties:
It is water soluble at room temperature, i.e. at 25° C. In preferred embodiments, the extracts also are soluble at lower temperatures as well (such as 15° C., 10° C. or even as low as 4° C., although more stirring over a longer period of time may be required). The WSTE contains substantially no, or only negligible quantities of lycopene (less than 0.5% by dry weight, preferably less than 0.1% by dry weight).
It is substantially free from water-insoluble particulate material (i.e. less than 0.1% by dry weight, preferably less than 0.01% by dry weight of particulate material).
It may be in liquid or dry forms. In some forms, such as in the dry extract FRUITFLOW 2, the sugars have been removed, and the extract has been concentrated.

Particulate Air Pollution: This is air pollution which contains particles which are classified as nanoparticles, or have a particle size of PM2.5 or less. These size particles can be the result of “natural sources” such as volcanic emission, dust storms, forest fires, smoke from grassland fires and the like, or as a result of human activity such as automotive emissions, manufacturing emissions or other activities, including smoking.

Cardiovascular health: This term is defined as conditions associated with unwanted platelet aggregation, such as: arthrosclerosis, myocardial infarction, stroke, thrombosis, peripheral artery disease, or decreased cerebral blood flow, and also includes diabetes (Type 1 or Type 2) and its associated cardiovascular problems.

Healthy Person—for purposes of this invention, a healthy person has not been diagnosed with, nor is aware of any cardiovascular health problems which are related to unwanted platelet aggregation, arthrosclerosis, myocardial infarction, stroke, thrombosis, peripheral artery disease, decreased cerebral blood flow, or diabetes (either Type 1 or Type 2).

### Preferred Compositions

A preferred WSTE comprises a tomato extract which is substantially lycopene-free, substantially heat stable and comprises water soluble compounds that have activity for preventing platelet aggregation and which have a molecular weight of less than 1000 daltons.

Preferably the WSTE comprises a, some or each of the water soluble compounds with activity for preventing platelet aggregation selected from the group comprising:
(a) glycosylated phenolic acids or phenolic esters, or derivatives thereof
(b) glycosylated flavonoids; and
(c) nucleosides.

It is preferred that the WSTE used according to the invention is a tomato extract described in WO2010/049707. Preferably the WSTE is made according to the methods described in WO2010/049707. For instance, in some embodiments the WSTE may be made according to the methods described in FIG. 2 or 4 of WO2010/049707.

In one embodiment the WSTE is a liquid in the form of a syrup.

A preferred extract has a browning index of <0.8 AU, a pH of 4.0-4.3 and a density of 1.15-1.20 and may be prepared by the steps of:
(a) Preparing a start mix of homogenised tomato;
(b) Separating a water soluble fraction from fruit solids;
(c) filtration of the water soluble fraction to make the extract substantially lycopene-free; and
(d) concentration of water soluble compounds with activity for preventing platelet aggregation in the filtration permeate using an evaporator.

In another embodiment sugars may be removed from the extract. It is preferred that such extracts contain <1% sugar, and contain >95% of the water soluble compounds with activity for preventing platelet aggregation that are contained in a start mix of homgenised tomatoes from which the WSTE is derived. Such extracts may be in the form of concentrated aqueous solutions or preferably in powder form. In a most preferred embodiment such an extract may be made by the steps of:
(a) Preparing a start mix of homogenised tomato fruit, wherein the pH of the start mix does not exceed pH 5.5, the holding temperature of the start mix does not exceed 35° C. and the start mix is diluted with water such that it comprises less than 33% solids;
(b) Separating a water soluble fraction from fruit solids by a procedure that does not raise the temperature of the fraction above 60° C.;
(c) filtration of the water soluble fraction;
(d) removal of free sugars from the filtered water soluble fraction; and
(e) concentration of water soluble compounds with activity for preventing platelet aggregation by a procedure that does not raise the temperature of the fraction above 60° C.;

Following step (e) the extract may be a concentrated aqueous solution containing <1% sugar, and containing >95% of water soluble compounds with activity for preventing platelet aggregation contained in the start mix. Such aqueous solutions may be dehydrated further to form a powder.

In some embodiments the WSTE may be provided in a composition that contains other molecules that are beneficial to human health. For instance, the composition may also contain nitrate or a precursor of nitric oxide. The nitrate is preferably from a source of dietary nitrate (for instance, and purely by way of example, a water-soluble extract from swiss chard, rocket, spinach, rhubarb, strawberry or lettuce). The composition may also comprise folic acid or a metabolite thereof (e.g. 5-methoxytetrahydrofolate or tetrahydrofolate). Preferred compositions for use according to the present invention which comprise folic acid or a metabolite thereof and/or nitrate are described in WO2014/102546.

Subjects that benefit from WSTE treatment are preferably human subjects. The inventors have found that healthy persons and those with a pre-existing cardiovascular condition can benefit from taking WSTE if they are at risk of being exposed to particulate air pollution.

### Use in Healthy Persons

In some embodiments the person is a healthy person. In preferred embodiments where the person ingests the WSTE of this invention prior to exposure to air pollution, the ingestion occurs at least 30 minutes to 1 hour prior to the exposure. In particularly preferred embodiments, the ingestion occurs at least 2 or at least 3 hours prior to exposure in order to ensure the food or food supplement containing the WSTE has been digested and that the WSTE active ingredients have entered the circulatory system at their optimum levels. In areas where air pollution occurs in sustained episodes (i.e. air pollution lasts more than one day), the WSTE should be taken prior to the first episode and at least daily during the air pollution episode. In other embodiments, the WSTE is taken at least daily during the portion of the year where air pollution episodes are likely to occur.

Another embodiment of this invention is a method of maintaining healthy blood flow, or minimizing the risk of platelet aggregation in a person exposed to particulate air pollution comprising administering to the person at risk of exposure an effective amount of WSTE prior to or during exposure to particulate air pollution.

In another aspect of this invention the person at risk generally enjoys good cardiovascular health, i.e. does not have known problems associated with cardiovascular health.

Another embodiment of this invention is the use of WSTE to non-therapeutically maintain healthy blood flow in a healthy person at risk of exposure to particulate matter type air pollution. Examples of non-therapeutic results include: decreasing the risk of appearing older due to skin care issues, particularly wrinkles or hardening of the skin, and/or maintaining general well-being and balance of energies due to good blood circulation.

Other uses of the WSTE of this invention include:
Maintaining healthy platelet function in the presence of air pollution;
Maintaining a healthy blood circulation and blood flow in the presence of air pollution;
Reducing the risk of an adverse cardiovascular condition, such as atherosclerosis, or thrombosis in the presence of particulate matter air pollution;
Reducing the severity of cardiovascular diseases when exposed to particulate matter; and
Reducing the risk of cardiovascular and respiratory illness in an air polluted environment.
   Use in Persons Who Already have Cardiovascular Disease

It has also been surprisingly found that WSTE shows a synergistic anti-platelet aggregation effect in the presence of both adenosine diphosphate (ADP) and PM. ADP, a natural platelet agonist stored in platelets, is released upon platelet activation; and it induces a strong initiation of platelet aggregation. PM also strongly induces platelet aggregation generally, and also further promotes the platelet aggregation induced by ADP. The combination of ADP plus PM is inducing a stronger platelet activation response than the sum of the individual platelet activation responses induced by ADP or PM alone. As shown in more detail in the Examples, we have found that WSTE inhibits both the platelet activity induced by PM present in air pollution, and surprisingly it also inhibits the ADP induced platelet aggregation when promoted by the presence of PM.

Thus the WSTE of this invention, if desired, can also be used in a population of people who have a history of cardiovascular health problems or diabetes, and therefore have ADP present in their circulating blood, and are also at risk of exposure to particulate air pollution. Thus, another embodiment of this invention is a method of maintaining cardiovascular health in persons who have a history of cardiovascular health problems and who are exposed to particulate air pollution or who are at risk of exposure to particulate air pollution comprising administering an effective amount of a WSTE extract to the person prior to exposure or during exposure to the particulate air pollution.

Another aspect of this invention is the use of WSTE to protect the user against the harmful cardiovascular effects of air pollution, preferably particulate matter air pollution. A person who is exposed to such air pollution or who is at risk of exposure to such air pollution can ingest WSTE and thereby protect him/herself against cardiovascular problems associated with air pollution.

Another aspect of this invention is a method of decreasing the risk of cardiovascular health problems associated with particulate air pollution induced platelet aggregation comprising administering WSTE to a person at risk of exposure to particulate air pollution.

Another aspect of this invention is the use of water soluble tomato extract for the use in manufacturing a pharmaceutical or nutraceutical capable of maintaining cardiovascular health, lessening the risk of developing a cardiovascular health problem, or reducing the likelihood of worsening an existing cardiovascular health problem in a person who is exposed or is at risk of exposure to particulate air pollution.

### Dosages and Formulations

### Doses:

Tomato Extract: Preferably, FRUITFLOW® 2 (a powder form) is used, although FRUITFLOW 1 (a liquid form) may be preferable if the formulation is to be liquid. The amount of WSTE should be from 25-300 mg/day preferably from 75-200 mg/day, and more preferably 125-175 mg/day. In some embodiments, 100 or 150 mg/day may be consumed by a subject. The aforementioned amounts may be taken as a single once-a-day dose, or partial dosages may be taken more than once a day (i.e. 2 or 3 times per day) so that the full dose is consumed. Preferred dosage forms according to the invention comprise 25-300 mg of WSTE, preferably 75-200 mg of WSTE and more preferably 125-175 mg of WSTE.

Timing of the dosage: It is preferable to consume WTSE prior to exposure to the air pollution episode, preferably at least 2-3 hours prior, so that the WTSE is properly metabolized and is available in the circulatory system prior to the exposure. In other embodiments, the ingestion occurs at least 30 minutes to 1 hour prior to the exposure. In areas where air pollution occurs in sustained episodes (i.e. air pollution lasts more than one day), the WSTE should be taken prior to the first episode and at least daily during the air pollution episode. In other embodiments, the WSTE is taken at least daily during the portion of the year where air pollution episodes are likely to occur.

### Forms

In one embodiment the compositions of the invention may be in the form of a nutraceutical. The term “nutraceutical” as used herein denotes usefulness in both nutritional and pharmaceutical fields of application. Thus, nutraceutical compositions comprising WSTE can be used as supplements to food and beverages and as pharmaceutical formulations for enteral or parenteral application which may be solid formulations, such as capsules or tablets, or liquid formulations, such as solutions or suspensions.

The WSTE nutraceutical compositions according to the present invention may further contain protective hydrocolloids (such as gums, proteins, modified starches), binders, film-forming agents, encapsulating agents/materials, wall/shell materials, matrix compounds, coatings, emulsifiers, surface active agents, solubilising agents (oils, fats, waxes, lecithins etc.), adsorbents, carriers, fillers, co-compounds, dispersing agents, wetting agents, processing aids (solvents), flowing agents, taste-masking agents, weighting agents, gelling agents, gel-forming agents, antioxidants and antimicrobials.

The nutraceutical compositions according to the present invention may be in any galenic oral form containing a conventional carrier material that is suitable for administering to the body, e.g. in solid forms such as (additives/supplements for) food, food premix, fortified food, tablets, pills, granules, dragées, capsules and effervescent formulations, such as powders and tablets, or in liquid forms, such as solutions, emulsions or suspensions as e.g. beverages, pastes and oily suspensions. The pastes may be incorporated in hard- or soft-shell capsules, whereby the capsules feature e.g. a matrix of animal-derived gelatin, plant proteins or ligninsulfonate.

If the nutraceutical composition is a pharmaceutical formulation the composition further contains pharmaceutically acceptable excipients, diluents or adjuvants. Standard techniques may be used for their formulation, as e.g. disclosed in Remington's Pharmaceutical Sciences, 20th edition Williams & Wilkins, PA, USA. For oral administration, tablets and capsules are preferably used which contain a suitable binding agent, e.g. gelatine or polyvinyl pyrrolidone, a suitable filler, e.g. lactose or starch, a suitable lubricant, e.g. magnesium stearate, and optionally further additives.

In some embodiments the compositions may be formulated for consumption as a food or drink. Examples of such foods or drinks are dairy products including, for example, margarines, spreads, butter, cheese, yoghurts or milk-drinks.

Other examples of foods that may be fortified with WSTE include bread, cereal bars, bakery items, such as cakes and cookies, and potato chips or crisps.

Beverages encompass non-alcoholic and alcoholic drinks as well as liquid preparations to be added to drinking water and liquid food. Non-alcoholic drinks are e.g., soft drinks, sports drinks, fruit juices, lemonades, teas and milk-based drinks. Liquid foods are e.g., soups and dairy products.

Nutraceutical compositions containing WSTE may be added to a soft drink, an energy bar, or a candy.

The non-limiting examples are presented to further illustrate the invention.

### EXAMPLES

### Example 1

Reagents: Diesel Particulate Matter (Industrial Forklift, SRM2975) was from National Institute of Standards and Technology (Gaithersburg, MD, USA). Adenosine diphosphate (ADP), dimethyl sulfoxide (DMSO) and titanium (IV) oxide (TiO2) anatase were from Sigma (Saint-Louis, MO). Phosphate buffered saline (PBS) was from Invitrogen (Carlsbad, CA).

Preparation of diesel particulate matter and TiO2: Particles were suspended in DMSO and sonicated in a sonicating water bath for 5 min to minimize agglomeration. They were diluted at appropriate concentrations in PBS before use.

Platelet aggregation measurements: Blood from healthy human volunteers was collected through Safety-Multifly® needle into Sodium Citrate S-Monovette® tubes (Sarstedt, Nümbrecht, Germany). Platelet-rich plasma (PRP) was obtained by centrifugation of citrated blood at 150 g for 15 min at 37° C. PRP were transferred into plastic tubes and left at 37° C. Remaining blood was centrifuged at 2000 g for 15 min at 37° C. to obtain platelet-poor plasma (PPP). Platelet counts were determined using a Sysmex cell counter (Norderstedt, Germany) and the platelet number in PRP was adjusted to 3×10⁸ platelets/mL with autologous PPP. PRP were incubated with Fruitflow®2 (86 μg/mL) or PBS at 37° C. for 10 min prior to stimulation. The PRP suspensions were stimulated with ADP (2.5 M), diesel particulate matter (50 μg/mL) or TiO₂ (50 μg/mL) in the presence or absence of Fruitflow® and the platelet aggregation was monitored on a platelet aggregometer (APACT 4004, Labitec, Ahrensburg, Germany) for 10 min at 37° C. under stirring conditions. PPP was used to determine the baseline (100% aggregation). As particles can impact the light transmission, PPP with diesel particulate matter (50 μg/mL) or TiO₂ (50 μg/mL) was used as baseline in the presence of respectively diesel particulate matter or TiO₂. The platelet aggregation was quantified as area under the aggregation curve (AUC) and as the maximal percent aggregation (max % aggr.).

### Results

ADP is a natural platelet agonist stored in platelets and is released upon platelet activation; it induces a strong initiation of platelet aggregation. Diesel particulate matter (PM) also strongly induces platelet aggregation. In contrast, non-polluted TiO₂ particles did not induce significant aggregation at equivalent concentrations. Moreover PM further promoted the platelet aggregation induced by ADP. The combination of ADP plus PM is inducing a stronger platelet activation response than the sum of the individual platelet activation responses induced by ADP or PM alone.

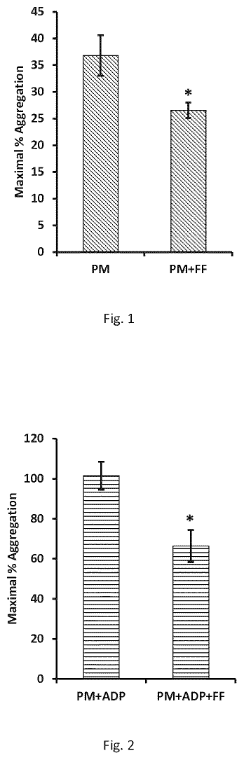

Interestingly Fruitflow® was also able to inhibit the platelet aggregation induced by PM. Thus the area under the aggregation curve (AUC) was significantly decreased by 30% from 18606 to 13079.and the maximal percent aggregation (max % aggr.) was significantly decreased by 28% from 37% to 27% in the presence of Fruitflow® (Table 1 and FIG. 1). Finally, the platelet aggregation which is promoted by the combination of ADP and PM is strongly inhibited by Fruitflow®. The area under the aggregation curve (AUC) was significantly decreased by 36% from 55622 to 35511. The maximal percent aggregation (max % aggr.) was significantly decreased by 35% from 101% to 66% in the presence of Fruitflow® (Table 1 and FIG. 2)

TABLE 1
Effect of Fruitflow ® on platelet aggregation induced by air pollution particulate matter

| | ADP | ADP + FF | PM | PM + FF | PM + ADP | PM + ADP + FF |
|---|---|---|---|---|---|---|
| AUC | 27953 ± 4856 | 6656 ± 1246* | 18606 ± 2207 | 13079 ± 683* | 55622 ± 3288 | 35511 ± 4297* |
| Max % aggr. | 54 ± 7 | 18 ± 5* | 37 ± 4 | 27 ± 1* | 101 ± 7 | 66 ± 8* |

All values are mean ± SD.
ADP, Adenosine diphosphate.
PM, particulate matter.
FF, Fruitflow ®.
AUC, area under the aggregation curve.
Max % aggr., maximal percent aggregation.
*p < 0.05 significantly different from the respective treatment not receiving FF (e.g. ADP vs. ADP + FF, PM vs PM + FF, PM + ADP vs PM + ADP + FF).

### CONCLUSION

Fruitflow® inhibits the platelet activity induced by PM present in air pollution. Moreover, Fruitflow® also inhibits the ADP induced platelet aggregation when promoted by the presence of PM.

Thus, Fruitflow® reduces the platelet activation induced by PM which promote arterial thrombosis, atherosclerosis, and other cardiovascular diseases. Fruitflow® may be particularly useful in case of already elevated platelet reactivity due to stress. Furthermore, the WTSE can be used in persons with a preexisting disease such as diabetes or cardiovascular disease as the platelet reactivity which is increased by natural platelet agonist released under stress conditions and disease such as ADP are further promoted by air pollutants such as PM and can be inhibited by Fruitflow® such reducing the risk of cardiovascular events induced by PM. In conclusion, Fruitflow® is able to reduce the deleterious effects of PM on the cardiovascular system.

1. A method of maintaining cardiovascular health, lessening the risk of developing cardiovascular health problems, or reducing the likelihood of worsening an existing cardiovascular health problem in a subject exposed, or is at risk of exposure, to particulate air pollution comprising administering a composition comprising a water soluble tomato extract (WSTE).

2. The method according to claim 1 for use in the form of a pharmaceutical, nutraceutical, food supplement or food.

3. The method according to claim 1 wherein the subject is a human subject.

4. The method according to claim 3 wherein the human subject is a healthy person.

5. The method according to claim 3 wherein the human subject is a person with an existing cardiovascular disease.

6. The method according to claim 1 wherein the subject is exposed, or is at risk of exposure, to particulate air pollution which is formed as a result of: volcanic emissions, dust storms, forest fires, smoke from grassland fires, automotive emissions, manufacturing emissions or smoking.

7. The method according to claim 1 wherein the WSTE comprises a tomato extract which is:
(i) substantially lycopene-free;
(ii) substantially heat stable; and
(ii) comprises water soluble compounds that have activity for preventing platelet aggregation and which have a molecular weight of less than 1000 daltons.

8. The method according to claim 7 for use wherein the WSTE comprises a, some or each of the water soluble compounds with activity for preventing platelet aggregation selected from:
(a) glycosylated phenolic acids or phenolic esters, or derivatives thereof;
(b) glycosylated flavonoids; and
(c) nucleosides.

9. The method according to claim 7 wherein the WSTE component has a browning index of <0.8 AU, a pH of 4.0-4.3 and a density of 1.15-1.20.

10. The method according to claim 7 wherein the WSTE component is a concentrated aqueous solution containing <1% sugar, and containing >95% of the water soluble compounds with activity for preventing platelet aggregation contained in a start mix of homgenised tomatoes from which the WSTE is derived.

11. The method according to claim 7 wherein the WSTE component contains <1% sugar, and contains >95% of the water soluble compounds with activity for preventing platelet aggregation contained in a start mix of homgenised tomatoes from which the WSTE is derived and which is in powder form.

12. The method according to claim 1 wherein the composition comprises a quantity of WSTE which is sufficient to decrease, or minimize the risk of, platelet aggregation in the subject exposed to particulate air pollution.

13. A method for protecting against the adverse effects of particulate air pollution comprising administering a dosage form comprising a medicament, nutraceutical, food supplement or food containing an effective amount of water soluble tomato extract (WSTE).

14. The method according to claim 13 wherein the dosage form comprises 25-300 mg of WSTE.

15. (canceled)

16. A composition comprising a water soluble tomato extract (WSTE), wherein the WSTE comprises a tomato extract which is:
(i) substantially lycopene-free;
(ii) substantially heat stable; and
(iii) comprises water soluble compounds that have activity for preventing platelet aggregation and which have a molecular weight of less than 1000 Daltons.

17. The composition of claim 16, wherein the WSTE is a concentrated aqueous solution containing <1% sugar, and containing >95% of the water soluble compounds with activity for preventing platelet aggregation contained in a start mix of homgenised tomatoes from which the WSTE is derived.

18. A method of producing a composition comprising a water soluble tomato extract (WSTE) of claim 16, comprising:
(a) preparing a start mix of homogenised tomato;
(b) separating a water soluble fraction from fruit solids;
(c) filtration of the water soluble fraction to make the extract substantially lycopene-free; and
(d) concentration of water soluble compounds with activity for preventing platelet aggregation in the filtration permeate using an evaporator.

19. The method of claim 18, further comprising removal of free sugars from the filtered water soluble fraction before concentrating the water soluble compounds.
